(19) United States
(12) Patent Application Publication
(10) Pub. No.: US 2021/0228657 A1
(43) Pub. Date: Jul. 29, 2021

(54) FOOD COMPOSITION AND PHARMACEUTICAL COMPOSITION USED FOR INCREASING EXERCISE PERFORMANCE AND AMELIORATING FATIGUE

(71) Applicant: GLAC BIOTECH CO., LTD., TAINAN CITY (TW)

(72) Inventors: Chi-Chang HUANG, Tainan City (TW); Wei-Ling CHEN, Tainan City (TW); Mon-Chien LEE, Tainan City (TW); Yi-Ju HSU, Tainan City (TW); Hsieh-Hsun HO, Tainan City (TW); Shin-Yu TSAI, Tainan City (TW); Wen-Yang LIN, Tainan City (TW); Yi Wei KUO, Tainan City (TW)

(21) Appl. No.: 17/229,644

(22) Filed: Apr. 13, 2021

Related U.S. Application Data
(63) Continuation-in-part of application No. 16/514,602, filed on Jul. 17, 2019, now abandoned.

(30) Foreign Application Priority Data
Apr. 23, 2019 (TW) 108114165

(51) Int. Cl. A61K 35/745; A61K 47/46; A61K 9/00; A61P 21/06; A23L 33/00; A23L 33/135

(52) U.S. Cl. CPC A61K 35/745; A61K 47/46; A61K 9/0056; A23V 2002/00; A23L 33/40; A23L 33/135; A23Y 2300/55; A61P 21/06

(57) ABSTRACT

An isolated lactic acid bacteria strain: Bifidobacterium longum subsp. longum OLP-01 strain for increasing exercise performance and ameliorating fatigue is disclosed. A variety of animal experiments have proved that OLP-01 not only effectively improves muscle strength and swimming endurance but also significantly reduces fatigue-related biochemical indicators, including blood lactate, blood urea nitrogen and the activity of creatine kinase.

### CROSS-REFERENCE TO RELATED APPLICATIONS

This is a continuation-in-part Application of U.S. patent application Ser. No. 16/514,602, filed Jul. 17, 2019. This continuation-in-part Application claims the benefit of the U.S. patent application Ser. No. 16/514,602.

### BACKGROUND OF THE INVENTION

### 1. Field of the Invention

The present invention relates to a food composition and pharmaceutical composition, particularly to a food composition and pharmaceutical composition used for increasing exercise performance and ameliorating fatigue.

### 2. Description of the Prior Art

Bustle and competition pressure of modern life usually fatigues or overstresses people, even endangering health. While a person is fatigued, his action or exercise performance may fail to achieve the normal level. Whether a person feel fatigued is a subjective feeling, normally related with tissue damage or energy insufficiency. Fatigue may be caused by exercise or other types of stress. The recovery time is dependent on the type of exercise/stress and the way of nutritional supply.

Probiotics are a type of microorganism or a combination of several types of microorganisms. While human beings or animals fed with probiotics, the quality of the intestinal microbiota will be improved. Probiotics must be able to act on the host and enhance the health of the host. According to the information of the Ministry of Health and Welfare, ROC, none probiotic product have existed among the products certified as able to ameliorate fatigue so far (https://consumer.fda.gov.tw/Food/InfoHealthFood.aspx?nodeID=162#).

In general, lactic acid bacteria are safe to human bodies. It has been a target the manufacturers are eager to achieve: finding out the lactic acid bacteria strains could improve exercise performance and ameliorate fatigue, and using the lactic acid bacteria strains to develop nutritional supplement products which are safety and long term used.

### SUMMARY OF THE INVENTION

The present invention provides a food composition and a pharmaceutical composition with active or inactive lactic acid bacteria strains, which can enhance exercise ability and body composition, thus can enhance exercise performance and ameliorate fatigue.

In one embodiment, the present invention provides a method for improving exercise performance and ameliorating fatigue comprising administering a food composition to a subject, wherein the food composition includes an isolated lactic acid bacteria strain having the active effect of improving exercise performance and ameliorating fatigue, wherein the lactic acid bacteria strain is a Bifidobacterium longum subsp. longum OLP-01 strain deposited in a Deposition No. CGMCC 17345 in China General Microbiological Culture Collection Center (CGMCC); and a physiologically-acceptable excipient, diluent, or carrier.

In another embodiment, the present invention provides a method for improving exercise performance and ameliorating fatigue comprising administering a pharmaceutical composition to a patient, wherein the pharmaceutical composition includes an isolated lactic acid bacteria strain having the active effect of improving exercise performance and ameliorating fatigue, wherein the lactic acid bacteria strain is a Bifidobacterium longum subsp. longum OLP-01 strain deposited in a Deposition No. CGMCC 17345 in China General Microbiological Culture Collection Center (CGMCC); and a pharmaceutically-acceptable excipient, diluent, or carrier.

Below, embodiments are described in detail in cooperation with the attached drawings to make easily understood the objectives, technical contents, characteristics and accomplishments of the present invention.

### BRIEF DESCRIPTION OF THE DRAWINGS

FIG. 1 is a bar chart schematically illustrating the effect of the OLP-01 strain of the present invention on the forelimb grip strength;

FIG. 2 is a bar chart schematically illustrating the effect of the OLP-01 strain of the present invention on the exhaustion time in an exhaustive swimming test;

FIG. 3 is a bar chart schematically illustrating the effect of the OLP-01 strain of the present invention on the blood glucose concentration after 10 minutes of swimming;

FIG. 4 is a bar chart schematically illustrating the effect of the OLP-01 strain of the present invention on the blood ammonia concentration after 10 minutes of swimming;

FIG. 5 is a bar chart schematically illustrating the effect of the OLP-01 strain of the present invention on the blood urea nitrogen concentration after 90 minutes swimming and rest for 60 minutes;

FIG. 6 is a bar chart schematically illustrating the effect of the OLP-01 strain of the present invention on the activity of creatine kinase after 90 minutes swimming and rest for 60 minutes;

FIG. 7 is a bar chart schematically illustrating the effect of the OLP-01 strain of the present invention on the glycogen levels in livers and muscles;

FIG. 8 presents a series of histopathological images showing the effect of the OLP-01 strain of the present invention on the tissues and organs; and

FIG. 9 presents a series of immunohistochemical images showing the effect of the OLP-01 strain of the present invention on the gastrocnemius muscles.

### DESCRIPTION OF THE PREFERRED EMBODIMENTS

The present invention will be described in detail with embodiments and attached drawings below. However, these embodiments are only to exemplify the present invention but not to limit the scope of the present invention. In addition to the embodiments described in the specification, the present invention also applies to other embodiments. Further, any modification, variation, or substitution, which can be easily made by the persons skilled in that art according to the embodiment of the present invention, is to be also included within the scope of the present invention, which is based on the claims stated below. Although many special details are provided herein to make the readers more fully understand the present invention, the present invention can still be practiced under a condition that these special details are partially or completely omitted. Besides, the elements or steps, which are well known by the persons skilled in the art, are not described herein lest the present invention be limited unnecessarily. Similar or identical elements are denoted with similar or identical symbols in the drawings. It should be noted: the drawings are only to depict the present invention schematically but not to show the real dimensions or quantities of the present invention. Besides, matterless details are not necessarily depicted in the drawings to achieve conciseness of the drawings.

The lactic acid bacteria strain mentioned in the specification is a Bifidobacterium longum subsp. longum OLP-01 strain (also called Bifidobacterium longum), which is screened from healthy human intestinal tracts. The freeze-dried culture of the strain has been deposited in China General Microbiological Culture Collection Center (CGMCC, Address: Institute of Microbiology Chinese Academy of Sciences, No. 1 West Beichen Road Chaoyang District Beijing China) since Mar. 18, 2019. The details thereof are listed in Table. 1.

TABLE 1
Data of Deposited Strain of Lactic Acid Bacteria

| Strain | Specie | Deposition No. | Deposition Date |
|---|---|---|---|
| OLP-01 | Bifidobacterium longum | CGMCC 17345 | Mar. 18, 2019 |
| | sub sp. longum | | |

The Bifidobacterium longum subsp. longum OLP-01 strain, which is listed in Table. 1 and deposited in Deposition No. CGMCC 17345, can enhance sport durability and body composition and thus can improve exercise performance and ameliorate fatigue capability.

The present invention provides a food composition, which comprises an isolated lactic acid bacteria strain having the active effect of improving exercise performance and ameliorating fatigue; and a physiologically-acceptable excipient, diluent, or carrier. The present invention also provides a pharmaceutical composition, which comprises an isolated lactic acid bacteria strain having the active effect of improving exercise performance and ameliorating fatigue; and a pharmaceutically-acceptable excipient, diluent, or carrier. The isolated lactic acid bacteria strain is a Bifidobacterium longum subsp. longum OLP-01 strain, which is deposited in Deposition No. CGMCC 17345 in China General Microbiological Culture Collection Center (CGMCC). In one embodiment, the isolated lactic acid bacteria strain may be an active strain or an inactivated strain.

In the embodiment of the food composition, the physiologically-acceptable excipient, diluent or carrier may be a food. The food may be but is not limited to be a milk-containing drink, tea, coffee, a chewing gum, a tooth-cleaning candy (such as an oral strip, a chewable tablet, or jelly sweets), or a combination thereof. The milk-containing drink may be fermented milk, yoghurt, cheese, or powdered milk. In the embodiment of the pharmaceutic composition, the pharmaceutic composition may be an oral agent, such as a tablet, a capsule, a solution, or a powder.

In the embodiment of the food composition or pharmaceutic composition, the number of the lactic acid bacteria strains is over 10⁶ CFU (Colony-Forming Unit), more preferably over 10¹⁰ CFU.

### Embodiment I: The Morphology and General Properties of the Lactic Acid Bacteria Strain of the Present Invention

The taxonomic characteristics of the strain are identified with the 16S rDNA sequencing analysis and the API bacterial identification system. The morphology and general properties of the strain are listed in Table. 2.

TABLE 2
the morphology and general properties of the lactic acid
bacteria strain of the present invention

| Strain | Morphology and characteristics |
|---|---|
| Bifidobacterium | 1. The colonies grown in MRS agar are in form |
| longum subsp. | of solid circles in white color. The bacterium |
| longum OLP-01 | body has a middle-size or longer rod-like shape, |
| strain | and two ends thereof sometimes have Y-shaped |
| | branches. |
| | 2. They are gram-positive bacilli, unlikely to |
| | generate spores, free of catalase, oxidase and |
| | motility, being obligate anaerobic bacteria, and |
| | most suitable to grow at a temperature of |
| | 37 ± 1° C. |

In the experiments of the present invention, the administered dosage is based on the daily recommended dosage of 1×10¹⁰ CFU for a 60 kg adult. According to the method for “Estimating the maximum safe starting dose in initial clinical trials for therapeutics in adult healthy volunteers” (US FDA, 2005), the conversion coefficient of human body to mouse is 12.3. The 1-fold dose group was fed by a dosage of 2.05×10⁹ CFU/kg/day. The 2-fold dose group was fed by a dosage of 4.10×10⁹ CFU/kg/day. The 5-fold dose group was fed by a dosage of 1.03×10¹⁰ CFU/kg/day. The control (vehicle) group was fed with the same volume of phosphate buffered saline (PBS).

For animal experiments, forty 6-week-old ICR male mice were purchased from BioLASCO. Let these mice ingest water and animal fodder (Chow5001) freely. The animal rooms were controlled to be at a temperature of 24±2° C., a humidity of 60-70%, and with an illumination time of 12 hours and a dark time of 12 hours alternately. The mice were raised for two weeks to let them accustomed to the environment. Thus, these mice became 8 weeks old. Among these mice, select 40 ICR mice whose had about the same weight, and the selected mice were divided into 4 groups randomly: (a) the control (vehicle) group (Vehicle); (b) 1-fold dose group (OLP-01-1X) fed by a dosage of 2.05×10⁹ CFU/kg mouse/day; (c) 2-fold dose group (OLP-01-2X) fed by a dosage of 4.10×10⁹ CFU/kg mouse/day; (d) 5-fold dose group (OLP-01-5X) fed with a dosage of 1.03×10″ CFU/kg mouse/day.

TABLE 3
feed preparation in all groups

| | | | Demand for test material |
|---|---|---|---|
| | | | 0.3 mL test material was |
| | Volume | | tube-fed to a mouse each |
| | for unit | | time (such as a mouse |
| | body | | weighing 30 g); thus |
| | weight | | 0.3 × 10 = 3 mL was |
| Dosage | (mL/kg | Concentration | required for a group |
| (g/kg bw/day) | bw) | (CFU/mL) | each time. |
| Control group | 10 | 0 | 5 ml PBS |
| OLP-01-1X | 10 | 2.05 × 10⁸ | 1.03 × 10⁹ CFU + 5 ml |
| (2.05 × 10⁹CFU/ | | | PBS = 2.05 × 10⁸ CFU/mL |
| kg/day) | | | |
| OLP-01-2X | 10 | 4.10 × 10⁸ | 2.05 × 10⁹ CFU + 5 ml |
| (4.10 × 10⁹CFU/ | | | PBS = 4.10 × 10⁸ CFU/mL |
| kg/day) | | | |
| OLP-01-5X | 10 | 1.03 × 10⁹ | 5.15 × 10⁹ CFU + 5 ml |
| (10.3 × 10⁹CFU/ | | | PBS = 1.03 × 10⁹ CFU/mL |
| kg/day) | | | |

The duration of the animal experiment was 4 weeks. The four groups of mice were respectively tube-fed at 9:00 AM every day from the first week to the fourth week. After the mice had been supplied with the OLP-01 strain of the present invention for 4 weeks, the sport capabilities and fatigue-related biochemical indexes of the mice were analyzed. The experiments are designed without departing from the welfare of animals. The sport challenges were designed to test the strength from low to high, and interval time from short to long. Then, the blood of the mice were harvested for tests.

### Embodiment II: Animal Experiment for Mice Fed with the Lactic Acid Bacteria Strain of the Present Invention: Forelimb Grip Test

The forelimb grip strengths were measured to evaluate the effect of different dosages of the OLP-01 strain of the present invention on the mice. The experiment was designed to learn the extend of muscle strength increasing based on the experiment reported by Huang, Wu and Yeh (Huang W C, Lin C I, Chiu C C, Lin Y T, Huang W K, Huang H Y, Huang C C. (2014). Chicken essence improves exercise performance and ameliorates physical fatigue. Nutrients. 6(7):2681-2696); Wu R E, Huang W C, Liao C C, Chang Y K, Kan N W, Huang C C. (2013). Resveratrol protects against physical fatigue and improves exercise performance in mice. Molecules 18(4):4689-4702); Yeh T S, Chuang H L, Huang W C, Chen Y M, Huang C C, Hsu M C. (2014). Astragalus membranaceus Improves Exercise Performance and Ameliorates Exercise-Induced Fatigue in Trained Mice. Molecules 19(3):2793-2807). On the 29th day, the forelimb grip experiment was undertaken 30 minutes later after the mice of the experimental groups were fed with the OLP-01 strain of the present invention.

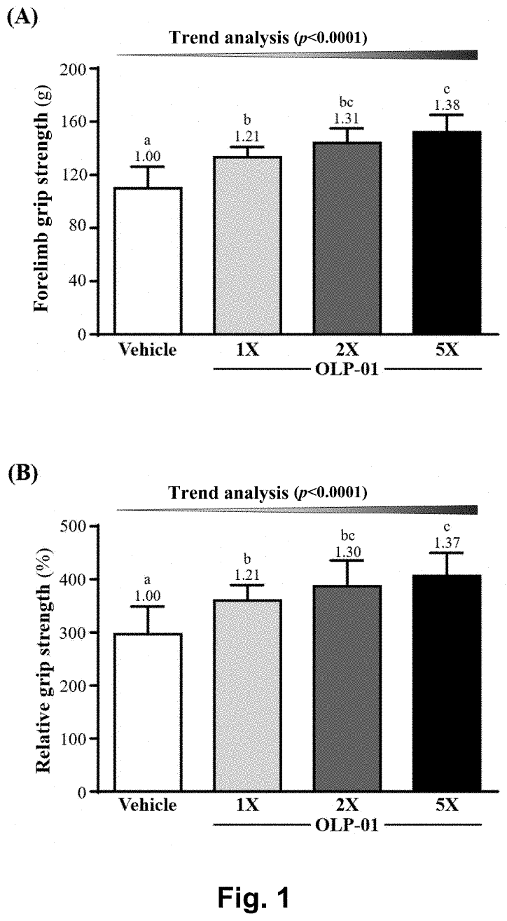

FIG. 1(A) shows the forelimb grip strengths of the vehicle group, the OLP-01-1X group, the OLP-01-2X group and the OLP-01-5X group, which were supplied with the OLP-01 strain of the present invention, wherein the forelimb grip strengths of the four groups are respectively 110±16 g, 133±8 g, 144±11 g and 152±13 g. The different English letters (a, b, c) in the above of the bar chart denote that there is significant difference (p<0.05). In comparison with the vehicle group, the forelimb grip strengths of the OLP-01-1X group, the OLP-01-2X group and the OLP-01-5X group, which were supplied with the OLP-01 strain of the present invention, respectively significantly increase 1.21 times (p=0.0002), 1.31 times (p<0.0001), and 1.38 times (p<0.0001). The trend analysis (p<0.0001) also indicates that the forelimb grip strength significantly increases with the dosage of the OLP-01 strain.

The body weight of an individual animal may influence the grip strength thereof. In order to eliminate the influence of the individual body weight on the forelimb grip strength, the relative grip strength of each group is further examined, wherein the relative grip strength=the forelimb grip strength/body weight x 100%. As shown in FIG. 1(B), the relative forelimb grip strengths of the vehicle group, the OLP-01-1X group, the OLP-01-2X group and the OLP-01-5X group are respectively 297±52(%), 360±29(%), 387±49(%) and 406±44(%). The different English letters (a, b, c) in the above of the bar chart denote that there is significant difference (p<0.05). In comparison with the vehicle group, the relative forelimb grip strengths of the OLP-01-1X group, the OLP-01-2X group and the OLP-01-5X group respectively significantly increase 1.21 times (p=0.0029), 1.30 times (p<0.0001), and 1.37 times (p<0.0001). The trend analysis (p<0.0001) also indicates that the relatively forelimb grip strength significantly increases with the dosage of the OLP-01 strain.

### Embodiment III: Animal Experiment for Mice Fed with the Lactic Acid Bacteria Strain of the Present Invention: Performance of Endurance Exercise

The experiments are used to evaluate whether 4-week OLP-01 supply can improve the performance of endurance exercise. On the 21th day (one week before the experiment) and at the time point 30 minutes later after the mice of the experimental groups were fed with the OLP-01 strain of the present invention, let the animals swim in a container 28 cm in diameter and 25 cm in depth with a water temperature of 27±1° C. to accustom themselves to swimming. After 4-week OLP-01 supply, on the 31th day, exhaustive swimming tests with a burden of 5% body weight of a mouse (Wu et al., 2013) were undertaken. The mice were fasted for 12 hours before swimming. In the exhaustive swimming test, each mouse swam alone. The mouse was placed in a water container and forced to swim. The water temperature was kept within 27±1° C. During the entire test, the four limbs of the mouse must be kept moving. If the mouse floated on the water surface with the limbs thereof motionless, use a stirring rod to stir the surroundings of the mouse. The time was counted from the beginning until the head of the mouse did not surface from water but had been submerged into water persistently for 8 seconds.

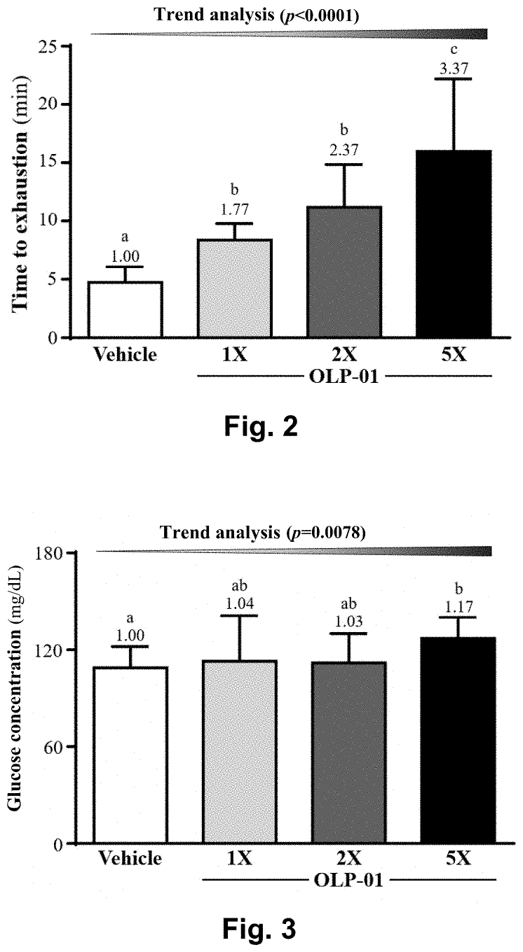

The results are shown in FIG. 2, wherein all the values are expressed in Mean±SD. In the exhaustive swimming tests, the exhaustion times of the vehicle group, the OLP-01-1X group, the OLP-01-2X group and the OLP-01-5X group are respectively 4.74±1.33 (min), 8.36±1.42 (min), 11.20±3.65 (min) and 15.98±6.20 (min). The different English letters (a, b, c) in the above of the bar chart denote that there is significant difference (p<0.05). In comparison with the vehicle group, the exhaustion times of the OLP-01-1X group, the OLP-01-2X group and the OLP-01-5X group, which are supplied with the OLP-01 strain of the present invention, respectively significantly increase 1.77 times (p=0.036), 2.37 times (p=0.0004) and 3.37 times (p<0.0001). The trend analysis (p<0.0001) also indicates that the exhaustion time significantly increases with the dosage of the OLP-01 strain.

### Embodiment IV: Animal Experiment for Mice Fed with the Lactic Acid Bacteria Strain of the Present Invention: Blood Lactate Analysis

In one embodiment, variation of blood lactate related with fatigue is examined. On the day of the swimming test (the 33th day), undertake blood harvesting of the mice 30 minutes later after the mice of the experimental groups were fed with the OLP-01 strain of the present invention. Next, let the mice swim without any burden in the water at a temperature of 27±1° C. for 10 minutes, and undertake blood harvesting immediately after swimming. Next, let the mice take a 20-minute rest, and then undertake blood harvesting. At the abovementioned three time points, 0.2 ml blood is harvested from each mouse each time for blood lactate analysis.

TABLE 4
the effect of supplying the OLP-01 strain of the present
invention on the increasing and decreasing of blood lactate concentration

| | | | | | Trend |
|---|---|---|---|---|---|
| Time | | | | | Anal- |
| point | Vehicle | OLP-01-1X | OLP-01-2X | OLP-01-5X | ysis |
| | Blood lactate (mmol/L) | | | | |
| Before | 3.42 ± 0.37 | 3.43 ± 0.36 | 3.41 ± 0.30 | 3.43 ± 0.24 | 0.7111 |
| swim- | | | | | |
| ming | | | | | |
| (A) | | | | | |
| After | 6.74 ± 0.28 ^{b} | 5.64 ± 0.57 ^{a} | 5.40 ± 0.83 ^{a} | 5.12 ± 0.59 ^{a} | <0.0001 |
| swim- | | | | | |
| ming | | | | | |
| (B) | | | | | |
| After | 6.05 ± 0.25 ^{b} | 5.04 ± 0.65 ^{a} | 4.76 ± 0.67 ^{a} | 4.53 ± 0.57 ^{a} | <0.0001 |
| 20-min | | | | | |
| rest | | | | | |
| (C) | | | | | |
| Pro- | 1.99 ± 0.23 ^{c} | 1.64 ± 0.05 ^{b} | 1.58 ± 0.12 ^{ab} | 1.49 ± 0.10 ^{a} | <0.0001 |
| duction | | | | | |
| rate = | | | | | |
| B/A | | | | | |
| Clear- | 0.10 ± 0.02 | 0.11 ± 0.06 | 0.12 ± 0.03 | 0.12 ± 0.02 | 0.0222 |
| ance | | | | | |
| rate = | | | | | |
| (B-C)/ | | | | | |
| B | | | | | |

All the values in Table. 4 are expressed in Mean±SD. Different English letters (a, b) in the above of the values of the same row indicate that there is significant difference.

After 10-minute swimming, the blood lactate concentrations of the vehicle group, the OLP-01-1X group, the OLP-01-2X group and the OLP-01-5X group are respectively 6.74±0.28 mmol/L, 5.64±0.57 mmol/L, 5.40±0.83 mmol/L and 5.12±0.59 mmol/L. In comparison with the vehicle group, the blood lactate concentrations of the OLP-01-1X group, the OLP-01-2X group and the OLP-01-5X group respectively significantly decrease 16.38% (p=0.0002), 19.82% (p<0.0001) and 24.02% (p<0.0001), as shown in Table. 4. The trend analysis (p<0.0001) also indicates that increasing the dosage of the OLP-01 strain can significantly decrease the post-swimming blood lactate concentrations. Therefore, supplying the OLP-01 strain of the present invention can obviously inhibit the increasing of the post-swimming blood lactate concentration.

The before-swimming blood lactate concentration and the after-10-minute-swimming blood lactate concentration can be used to calculate the production rate, and the results are shown in Table. 4. The lactate production rate of the vehicle group, the OLP-01-1X group, the OLP-01-2X group and the OLP-01-5X group are respectively 1.99±0.23, 1.64±0.05, 1.58±0.12 and 1.49±0.10. In comparison with the vehicle group, the production rate of the OLP-01-1X group, the OLP-01-2X group and the OLP-01-5X group respectively significantly decrease 17.52%, 20.84% and 25.20% (p<0.0001 in the three groups). The trend analysis (p<0.0001) also indicates that increasing the dosage of the OLP-01 strain can significantly decrease the production rate.

The blood lactate concentration of the mouse having taken after a 20-minute rest that follows a 10-minute swimming is also analyzed. The blood lactate concentrations after a 20-minute rest of the vehicle group, the OLP-01-1X group, the OLP-01-2X group and the OLP-01-5X group are respectively 6.05±0.25 mmol/L, 5.04±0.65 mmol/L, 4.76±0.67 mmol/L and 4.53±0.57 mmol/L. In comparison with the vehicle group, the post-20-minute rest blood lactate concentrations of the OLP-01-1X group, the OLP-01-2X group and the OLP-01-5X group respectively significantly decrease 16.79% (p=0.0003), 21.30% (p<0.0001) and 25.14% (p<0.0001), as shown in Table. 4. The trend analysis (p<0.0001) also indicates that increasing the dosage of the OLP-01 strain can significantly decrease the post-20-minute rest blood lactate concentration. Therefore, supplying the OLP-01 strain of the present invention can obviously reduce the post-20-minute rest blood lactate concentration.

The post-swimming blood lactate concentration and the post-20-minute rest blood lactate concentration are used to calculate the clearance rate. The lactate clearance rate of the vehicle group, the OLP-01-1X group, the OLP-01-2X group and the OLP-01-5X group are respectively 0.10±0.02, 0.11±0.06, 0.12±0.03 and 0.12±0.02. In comparison with the vehicle group, the lactate clearance rate of the OLP-01-1X group, the OLP-01-2X group and the OLP-01-5X group respectively increase 1.1 times (p=0.7712), 1.2 times (p=0.4174) and 1.2 times (p=0.3852). Although the increase of the lactate clearance rate of each group does not reach a level of significance, the trend analysis (p=0.0222) still indicates that increasing the dosage of the OLP-01 strain can significantly increase the lactate clearance rate.

Besides, the mice of the experimental groups were supplied with the OLP-01 strain of the present invention 10 minutes later after the exhaustive swimming test, and the variation of the glucose concentrations was examined. The results are shown in FIG. 3, and all the values are expressed in Mean±SD. The glucose concentrations of the vehicle group, the OLP-01-1X group, the OLP-01-2X group and the OLP-01-5X group are respectively 109±13 mg/dL, 113±28 mg/dL, 112±18 mg/dL and 127±13 mg/dL. The different English letters (a, b) in the above of the bar chart denote that there is significant difference (p<0.05). In comparison with the vehicle group, the glucose concentrations of the OLP-01-1X group, the OLP-01-2X group and the OLP-01-5X group respectively significantly increase 1.04 times (p=0.6179), 1.03 times (p=0.7016) and 1.17 times (p=0.0411). The trend analysis (p=0.0078) also indicates that increasing the dosage of the OLP-01 strain can significantly increase the post-swimming glucose concentrations. Therefore, supplying the OLP-01 strain of the present invention can obviously increase the post-sport glucose concentration.

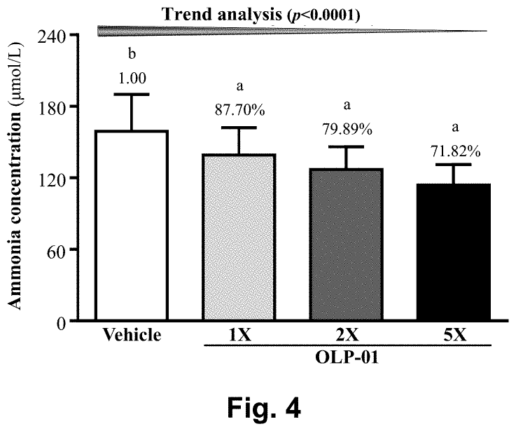

Further, the mice of the experimental groups were supplied with the OLP-01 strain of the present invention 10 minutes later after a single swimming sport, and the variation of the blood ammonia concentrations was examined. The results are shown in FIG. 4, and all the values are expressed in Mean±SD. The blood ammonia concentrations of the vehicle group, the OLP-01-1X group, the OLP-01-2X group and the OLP-01-5X group are respectively 159±31 μmol/L, 139±23 μmol/L, 127±19 μmol/L and 114±17 μmol/L. The different English letters (a, b) in the above of the bar chart denote that there is significant difference (p<0.05). In comparison with the vehicle group, the blood ammonia concentrations of the OLP-01-1X group, the OLP-01-2X group and the OLP-01-5X group respectively significantly decrease 12.30% (p=0.0710), 20.11% (p=0.0044) and 28.18% (p=0.0001). The trend analysis (p<0.0001) also indicates that increasing the dosage of the OLP-01 strain can significantly decrease the post-swimming blood ammonia concentration. Therefore, supplying the OLP-01 strain of the present invention can obviously decrease the post-swimming blood ammonia concentration.

### Embodiment V: Animal Experiment for Mice Fed with the Lactic Acid Bacteria Strain of the Present Invention: BUN Analysis and CK Activity Analysis

In one embodiment, after a sport challenge, variation of blood urea nitrogen (BUN) concentration of the mice is examined. On the day of the test (the 35th day) and at the time point 30 minutes later after the mice of the experimental groups were fed with the OLP-01 strain of the present invention, let the mice swim without burden for 90 minutes, and then let them take a 60-minute rest. Next, 0.3 ml blood was drawn from each mouse for BUN concentration analysis. Further, a portion of the blood was used in creatine kinase (CK) activity analysis, which may function as the index of muscle damage (such as the experimental index analysis reported by Huang, Wang, Wu, Su and Yeh), whereby to learn whether the OLP-01 strain of the present invention can reduce the muscle damage caused by long-term exercise. The serum samples of this test were analyzed with an automatic blood analyzer (Hitachi 7060, Hitachi, Tokyo, Japan). Please also refer to the following papers: Triterpenoid-rich extract from Antrodia camphorata improves physical fatigue and exercise performance in mice. Evid Based Complement Alternat Med. 2012:364741); Wang S Y, Huang W C, Liu C C, Wang M F, Ho C S, Huang W P, Hou C C, Chuang H L, Huang C C. (2012). Pumpkin (Cucurbita moschata) fruit extract improves physical fatigue and exercise performance in mice. Molecules 17(10):11864-11876; Wu R E, Huang W C, Liao C C, Chang Y K, Kan N W, Huang C C. (2013). Resveratrol protects against physical fatigue and improves exercise performance in mice. Molecules 18(4):4689-4702); Su K Y, Yu C Y, Chen Y W, Huang Y T, Chen C T, Wu H F, Chen Y L. (2014). Rutin, a flavonoid and principal component of Saussurea involucrata, attenuates physical fatigue in a forced swimming mouse model. Int J Med Sci. 11(5):528-537; Yeh T S, Chuang H L, Huang W C, Chen Y M, Huang C C, Hsu M C. (2014). Astragalus membranaceus Improves Exercise Performance and Ameliorates Exercise-Induced Fatigue in Trained Mice. Molecules 19(3):2793-2807.

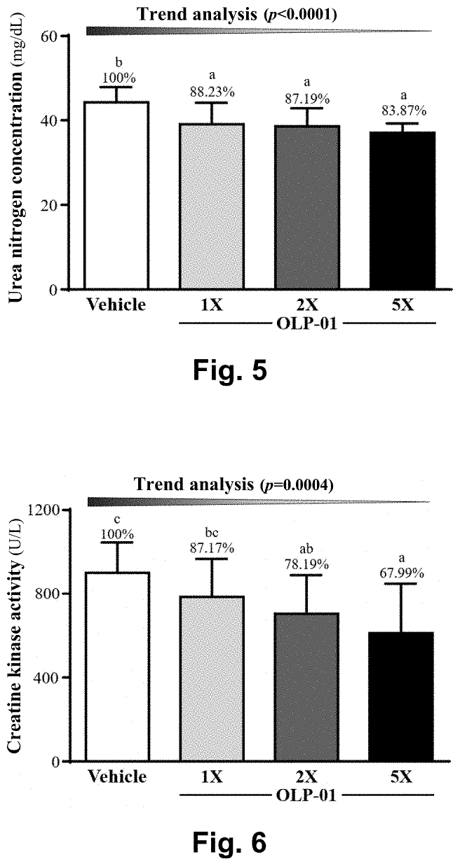

The results are shown in FIG. 5, wherein all the values are expressed in Mean±SD. The BUN concentrations of the vehicle group, the OLP-01-1X group, the OLP-01-2X group and the OLP-01-5X group are respectively 44.3±3.6 mg/dL, 39.1±5.1 mg/dL, 38.6±4.3 mg/dL and 37.1±2.2 mg/dL. The different English letters (a, b) in the above of the bar chart denote that there is significant difference (p<0.05). In comparison with the vehicle group, the BUN concentrations of the OLP-01-1X group, the OLP-01-2X group and the OLP-01-5X group respectively significantly decrease 11.77% (p=0.0054), 12.81% (p=0.0027) and 16.13% (p=0.0003). The trend analysis (p<0.0001) also indicates that increasing the dosage of the OLP-01 can significantly decrease the BUN concentration. Therefore, supplying the OLP-01 strain of the present invention can obviously decrease the post-swimming BUN concentration.

FIG. 6 shows the variation of the CK activity of each group of mice having persistently swum for 90 minutes without burden and then taken a 60-minute rest, wherein all the values are expressed in Mean±SD. The CK activities of the control (vehicle) group, the OLP-01-1X group, the OLP-01-2X group and the OLP-01-5X group are respectively 900±144 U/L, 784±182 U/L, 704±185 U/L and 612±236 U/L. The different English letters (a, b) in the above of the bar chart denote that there is significant difference (p<0.05). In comparison with the vehicle group, the CK activities of the OLP-01-1X group, the OLP-01-2X group and the OLP-01-5X group respectively significantly decrease 12.83% (p=0.1814), 21.81% (p=0.0264) and 32.01% (p=0.0017). The trend analysis (p=0.0004) also indicates that increasing the dosage of the OLP-01 strain can significantly decrease the CK activity.

### Embodiment VI: Animal Experiment for Mice Fed with the Lactic Acid Bacteria Strain of the Present Invention: Glycogen Analysis

Glycogen is an important energy storage material in animal bodies. In one embodiment, the effect of supplying the OLP-01 strain of the present invention on the level of glycogen is examined. On the 37th day (the day after a 2-day rest behind a 90-minute swimming), sacrifice the mice 30 minutes later after the final feeding, and collect blood samples for analysis. Further, collect, flush with normal saline, dry and weigh the livers and crural muscles of the hindlimbs of the mice. Next, take down the same regions of the tissues, package them separately, and then store them at a temperature of −80° C. for the succeeding glycogen analysis. The analysis was undertaken according to the chemical analysis method adopted by Chamberland and Rioux (Chamberland V, Rioux P. (2010). Not only students can express alcohol dehydrogenase: goldfish can too! Adv Physiol Educ 34(4):222-227). Take out the tissue samples, and add a tissue homogenate to each tissue sample by 5 times the volume of the tissue sample. Next, homogenize the tissue with a bullet blender (Next Advance, Cambridge, Mass., USA). Separately fill the homogenized liquids into microcentrifuge tubes, and centrifugalize them at a temperature of 4° C. and a centrifugal force of 12000×g for 15 minutes. Then, take the upper level of the extracted liquid, and undertake the glycogen analysis thereof with the method mentioned by Huang (Huang W C, Lin C I, Chiu C C, Lin Y T, Huang W K, Huang H Y, Huang C C. (2014). Chicken essence improves exercise performance and ameliorates physical fatigue. Nutrients. 6(7):2681-2696)). Further, use a commercially-available reference standard material of glycogen (Glycogen Sigma) to build a calibration curve, whereby to obtain the variation of the glycogen storage in the livers and muscles of the mice in the different groups.

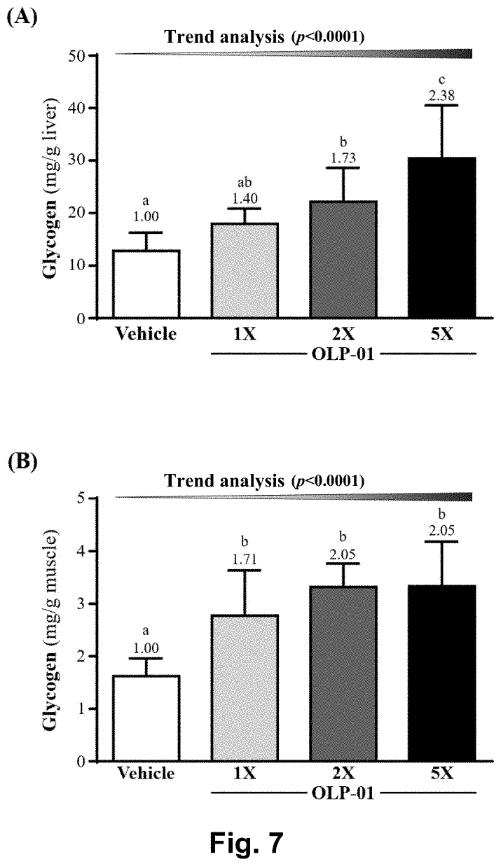

FIG. 7(A) shows the glycogen level in the livers of different groups, wherein all the values are expressed in Mean±SD. The glycogen levels in the livers of the vehicle group, the OLP-01-1X group, the OLP-01-2X group and the OLP-01-5X group are respectively 12.81±3.44 mg/g, 17.95±2.93 mg/g, 22.17±6.45 mg/g and 30.49±10.02 mg/g. The different English letters (a, b, c) in the above of the bar chart denote that there is significant difference (p<0.05). In comparison with the vehicle group, the glycogen levels in the livers of the OLP-01-1X group, the OLP-01-2X group and the OLP-01-5X group respectively significantly increase 1.40 times (p=0.0769), 1.73 times (p=0.0023) and 2.38 times (p<0.0001). The trend analysis (p<0.0001) also indicates that increasing the dosage of the OLP-01 strain can significantly increase the glycogen level in the liver.

FIG. 7(B) shows the glycogen levels in the muscles of the hindlimbs of different groups, wherein all the values are expressed in Mean±SD. The glycogen levels in the muscles of the vehicle group, the OLP-01-1X group, the OLP-01-2X group and the OLP-01-5X group are respectively 1.62±0.34 mg/g, 2.77±0.86 mg/g, 3.32±0.43 mg/g and 3.33±0.85 mg/g. The different English letters (a, b, c) in the above of the bar chart denote that there is significant difference (p<0.05). In comparison with the vehicle group, the glycogen levels in the muscles of the OLP-01-1X group, the OLP-01-2X group and the OLP-01-5X group respectively significantly increase 1.71 times (p=0.0004), 2.05 times (p<0.0001) and 2.05 times (p<0.0001). The trend analysis (p<0.0001) also indicates that increasing the dosage of the OLP-01 strain can significantly increase the glycogen level in the muscle.

### Embodiment VII: Animal Experiment for Mice Fed with the Lactic Acid Bacteria Strain of the Present Invention: Histopathological Analysis

In one embodiment, sacrifice four groups of mice 1 hour later after the final feeding, and section the tissues and organs thereof for histopathological analysis, whereby to learn whether the OLP-01 strain influences the tissues or organs unfavorably.

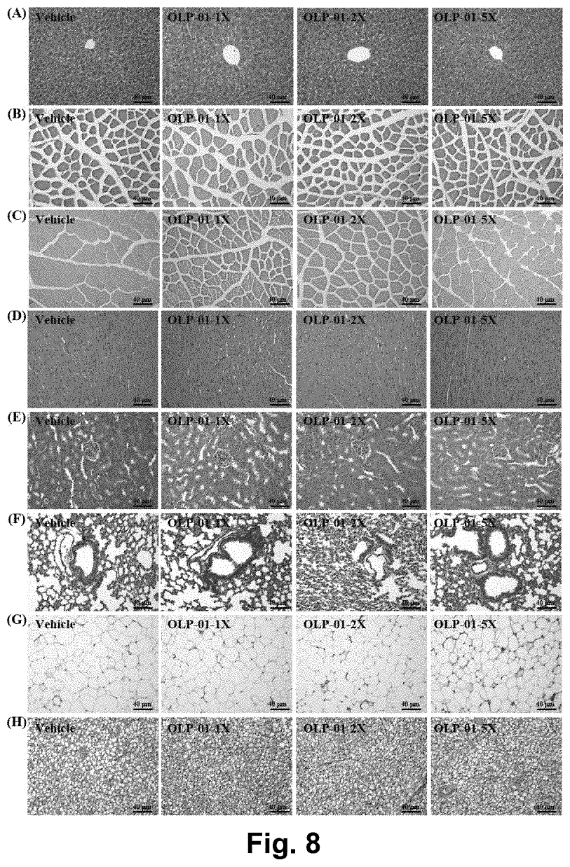

FIG. 8 shows that neither differences exist among nor pathological changes occur in the livers (A), muscles (B), quadriceps muscles (C), hearts (D), kidneys (E), lungs (F), epididymal fat pads (G), and brown adipose tissues (H).

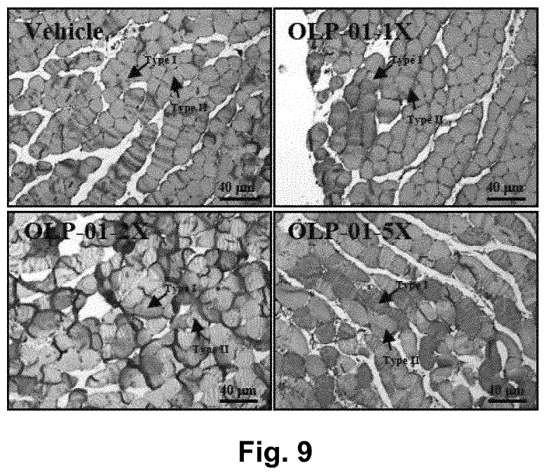

FIG. 9 shows that each of the OLP-01-1X group, the OLP-01-2X group and the OLP-01-5X group, which are supplied with the OLP-01 strain of the present invention, has more muscle fiber bundles in gastrocnemius muscle than the vehicle group. It is also found in the immunohistochemical images of muscles: each of the OLP-01-1X group, the OLP-01-2X group and the OLP-01-5X group has larger muscle fiber bundles than the vehicle group, and each of the OLP-01-1X group, the OLP-01-2X group and the OLP-01-5X group has more slow muscle fibers (Type I muscle fibers) than the vehicle group.

According to the abovementioned animal experiments, taking 1 time, 2 times or 5 times the daily recommended dosage (1×10¹⁰ CFU each person per day) of the OLP-01 strain of the present invention can significantly enhance muscle strength, prolong the exhaustion time in swimming with a burden, increase the blood lactate clearance rate, lower the BUN concentration, increase the glycogen storage in livers and muscles, improve the performance of endurance exercise, and postpone the occurrence of fatigue. It should be particularly explained: only few lactic acid bacteria strains are in-vivo experimentally proved to have the healthcare effect in ameliorating fatigue and improving exercise performance. It is the specificity of a strain but not the whole species of lactic acid bacteria that can favor human health. The strains having special effects on human health are called probiotics (Guidelines for the evaluation of probiotics in food; Report of joint FAO/WHO working group on drafting guidelines for the evaluation of probiotics in food; London Ontario, Canada April 30 and May 1, 2002:1-7). Therefore, what is claimed by the Inventors does not extensively cover all the strains of Bifidobacterium longum but is only the Bifidobacterium longum subsp. longum OLP-01 strain, which is deposited in a Deposition No. CGMCC 17345 in China General Microbiological Culture Collection Center (CGMCC).

The embodiments have been described above to demonstrate the technical thoughts and characteristics of the present invention to make the persons skilled in the art to understand, make, and use the present invention. However, these embodiments are not intended to limit the scope of the present invention. Any equivalent modification or variation according to the spirit of the present invention is to be also included by the scope of the present invention.

Bioresource Deposition

CGMCC 17345, China General Microbiological Culture Collection Center (CGMCC), Mar. 18, 2019

What is claimed is:

1. A method for improving exercise performance and ameliorating fatigue comprising administering a food composition to a subject, wherein the food composition comprises:
an isolated lactic acid bacteria strain having an active effect of improving exercise performance and ameliorating fatigue, wherein the lactic acid bacteria strain is a Bifidobacterium longum subsp. longum OLP-01 strain, which is deposited in a Deposition No. CGMCC 17345 in China General Microbiological Culture Collection Center (CGMCC); and
a physiologically-acceptable excipient, diluent, or carrier.

2. The method for improving exercise performance and ameliorating fatigue according to claim 1, wherein the lactic acid bacteria strain is an active strain.

3. The method for improving exercise performance and ameliorating fatigue according to claim 1, wherein the lactic acid bacteria strain is an inactivated strain.

4. The method for improving exercise performance and ameliorating fatigue according to claim 1, wherein the excipient, diluent or carrier is a food.

5. The method for improving exercise performance and ameliorating fatigue according to claim 4, wherein the food is fermented milk, yoghurt, cheese, milk-containing drink, powdered milk, tea, coffee, a chewing gum, a tooth-cleaning candy, or a combination thereof.

6. The method for improving exercise performance and ameliorating fatigue according to claim 1, wherein the exercise performance includes muscle strength and muscle endurance.

7. The method for improving exercise performance and ameliorating fatigue according to claim 6, wherein the muscle strength is forelimb grip strength.

8. The method for improving exercise performance and ameliorating fatigue according to claim 6, wherein the muscle endurance is endurance in swimming with a burden.

9. The method for improving exercise performance and ameliorating fatigue according to claim 1, wherein the active effect of ameliorating fatigue includes at least one of lowering blood lactate concentration, lowering blood ammonia concentration, lowering blood urea nitrogen concentration, lowering creatine kinase activity after exercise, and increasing glycogen storage in livers and muscles.

10. A method for improving exercise performance and ameliorating fatigue comprising administering a pharmaceutical composition to a patient, wherein the pharmaceutical composition comprises:
an isolated lactic acid bacteria strain having an active effect of improving exercise performance and ameliorating fatigue, wherein the lactic acid bacteria strain is a Bifidobacterium longum subsp. longum OLP-01 strain, which is deposited in a Deposition No. CGMCC 17345 in China General Microbiological Culture Collection Center (CGMCC); and
a pharmaceutically-acceptable excipient, diluent, or carrier.

11. The method for improving exercise performance and ameliorating fatigue according to claim 10, wherein the lactic acid bacteria strain is an active strain.

12. The method for improving exercise performance and ameliorating fatigue according to claim 10, wherein the lactic acid bacteria strain is an inactivated strain.

13. The method for improving exercise performance and ameliorating fatigue according to claim 10, which is in form of an oral agent.

14. The method for improving exercise performance and ameliorating fatigue according to claim 10, wherein the exercise performance includes muscle strength and muscle endurance.

15. The method for improving exercise performance and ameliorating fatigue according to claim 14, wherein the muscle strength is forelimb grip strength.

16. The method for improving exercise performance and ameliorating fatigue according to claim 14, wherein the muscle endurance is endurance in swimming with a burden.

17. The method for improving exercise performance and ameliorating fatigue according to claim 10, wherein the active effect of ameliorating fatigue includes at least one of lowering blood lactate concentration, lowering blood ammonia concentration, lowering blood urea nitrogen concentration, lowering creatine kinase activity after exercise, and increasing glycogen storage in livers and muscles.
